(19) United States
(12) Patent Application Publication
(10) Pub. No.: US 2023/0190451 A1
(43) Pub. Date: Jun. 22, 2023

(54) LENSES HAVING DIFFRACTIVE PROFILES WITH PARABOLIC TRANSITION ZONES

(71) Applicant: AMO Groningen B.V., Groningen (NL)

(72) Inventors: Aixa ALARCON HEREDIA, Groningen (NL); Miguel FARIA RIBEIRO, Braga (PT); Franck GOUNOU, Groningen (NL); Patricia A. PIERS, Groningen (NL); Hendrik WEEBER, Groningen (NL); Carmen CANOVAS VIDAL, Groningen (NL); Robert ROSEN, Groningen (NL)

(21) Appl. No.: 18/061,393

(22) Filed: Dec. 2, 2022

Related U.S. Application Data
(60) Provisional application No. 63/264,935, filed on Dec. 3, 2021.

(51) Int. Cl. A61F 2/16

(52) U.S. Cl. CPC A61F 2/1654; A61F 2/1618

(57) ABSTRACT

Apparatuses, systems and methods for providing improved ophthalmic lenses, particularly intraocular lenses (IOLs), include features for reducing adverse optical effects from diffractive profiles of such a lens. Exemplary ophthalmic lenses can include an optic including a diffractive profile including a transition zone having a parabolic shape.

### CROSS REFERENCE TO RELATED APPLICATIONS

This application claims the benefit under 35 U.S.C. § 119(e) of U.S. Provisional Application No. 63/264,935, filed Dec. 3, 2021, the contents of which are incorporated by reference as if fully set forth.

### BACKGROUND

Embodiments of the present disclosure relate to vision treatment techniques and in particular, to ophthalmic lenses such as, for example, contact lenses, corneal inlays or onlays, or intraocular lenses (IOLs) including, for example, phakic IOLs and piggyback IOLs (i.e. IOLs implanted in an eye already having an IOL).

Presbyopia is a condition that affects the accommodation properties of the eye. As objects move closer to a young, properly functioning eye, the effects of ciliary muscle contraction and zonular relaxation allow the lens of the eye to change shape, and thus increase its optical power and ability to focus at near distances. This accommodation can allow the eye to focus and refocus between near and far objects.

Presbyopia normally develops as a person ages and is associated with a natural progressive loss of accommodation. The presbyopic eye often loses the ability to rapidly and easily refocus on objects at varying distances. The effects of presbyopia usually become noticeable after the age of 45 years. By the age of 65 years, the crystalline lens has often lost almost all elastic properties and has only a limited ability to change shape.

Along with reductions in accommodation of the eye, age may also induce clouding of the lens due to the formation of a cataract. A cataract may form in the hard central nucleus of the lens, in the softer peripheral cortical portion of the lens, or at the back of the lens. Cataracts can be treated by the replacement of the cloudy natural lens with an artificial lens. An artificial lens replaces the natural lens in the eye, with the artificial lens often being referred to as an intraocular lens or “IOL.”

Monofocal IOLs are intended to provide vision correction at one distance only, usually the far focus. At the very least, since a monofocal IOL provides vision treatment at only one distance and since the typical correction is for far distance, spectacles are usually needed for good vision at near distances and sometimes for good vision at intermediate distances. The term “near vision” generally corresponds to vision provided when objects are at a distance from the subject eye at equal; or less than 1.5 feet. The term “distant vision” generally corresponds to vision provided when objects are at a distance of at least about 5-6 feet or greater. The term “intermediate vision” corresponds to vision provided when objects are at a distance of about 1.5 feet to about 5-6 feet from the subject eye. Such characterizations of near, intermediate, and far vision correspond to those addressed in Morlock R, Wirth R J, Tally S R, Garufis C, Heichel C W D, Patient-Reported Spectacle Independence Questionnaire (PRSIQ): Development and Validation. Am J Ophthalmology 2017; 178:101-114.

There have been various attempts to address limitations associated with monofocal IOLs. For example, multifocal IOLs have been proposed that deliver, in principle, two foci, one near and one far, optionally with some degree of intermediate focus. Such multifocal, or bifocal, IOLs are intended to provide good vision at two distances, and include both refractive and diffractive multifocal IOLs. In some instances, a multifocal IOL intended to correct vision at two distances may provide a near (add) power of about 3.0 or 4.0 diopters.

Multifocal IOLs may, for example, rely on a diffractive optical surface to direct portions of the light energy toward differing focal distances, thereby allowing the patient to clearly see both near and far objects. Multifocal ophthalmic lenses (including contact lenses or the like) have also been proposed for treatment of presbyopia without removal of the natural crystalline lens. Diffractive optical surfaces, either monofocal or multifocal, may also be configured to provide reduced chromatic aberration.

Diffractive monofocal and multifocal lenses can make use of a material having a given refractive index and a surface curvature which provide a refractive power. Diffractive lenses have a diffractive profile which confers the lens with a diffractive power that contributes to the overall optical power of the lens. The diffractive profile is typically characterized by a number of diffractive zones. When used for ophthalmic lenses these zones are typically annular lens zones, or echelettes, spaced about the optical axis of the lens. Each echelette may be defined by an optical zone, a transition zone between the optical zone and an optical zone of an adjacent echelette, and an echelette geometry. The echelette geometry includes an inner and outer diameter and a shape or slope of the optical zone, a height or step height, and a shape of the transition zone. The surface area or diameter of the echelettes largely determines the diffractive power(s) of the lens and the step height of the transition between echelettes largely determines the light distribution between the different powers. Together, these echelettes form a diffractive profile.

A multifocal diffractive profile of the lens may be used to mitigate presbyopia by providing two or more optical powers; for example, one for near vision and one for far vision. The lenses may also take the form of an intraocular lens placed within the capsular bag of the eye, replacing the original lens, or placed in front of the natural crystalline lens. The lenses may also be in the form of a contact lens, most commonly a bifocal contact lens, or in any other form mentioned herein.

Although multifocal ophthalmic lenses lead to improved quality of vision for many patients, additional improvements would be beneficial. For example, some pseudophakic patients experience undesirable visual effects (dysphotopsia), e.g. glare or halos. Halos may arise when light from the unused focal image creates an out-of-focus image that is superimposed on the used focal image. For example, if light from a distant point source is imaged onto the retina by the distant focus of a bifocal IOL, the near focus of the IOL will simultaneously superimpose a defocused image on top of the image formed by the distant focus. This defocused image may manifest itself in the form of a ring of light surrounding the in-focus image, and is referred to as a halo. Another area of improvement revolves around the typical bifocality of multifocal lenses. While multifocal ophthalmic lenses typically provide adequate near and far vision, intermediate vision may be compromised.

A lens with an extended range of vision may thus provide certain patients the benefits of good vision at a range of distances, while having reduced or no dysphotopsia. Various techniques for extending the depth of focus of an IOL have been proposed. One technique is embodied in the Tecnis Symfony® lens offered by Johnson & Johnson Vision. One technique may include a bulls-eye refractive principle, and may involve a central zone with a slightly increased power. One technique may include an asphere or include refractive zones with different refractive zonal powers.

Although certain proposed treatments may provide some benefit to patients in need thereof, further advances would be desirable. For example, it would be desirable to provide improved IOL systems and methods that confer enhanced image quality across a wide and extended range of foci without dysphotopsia. Further, improved IOL systems and methods to reduce visual symptoms produced by transition zones of diffractive profiles such as halo, glare, and scatter, may be desired. Embodiments of the present disclosure may provide solutions that address the problems described above, and hence may provide answers to at least some of these outstanding needs.

### BRIEF SUMMARY

Embodiments herein described include ophthalmic lenses including an optic. The optic may include a diffractive profile including at least one echelette having an optical zone and a transition zone, the transition zone having a parabolic shape. The echelette(s) have a width in r-squared space and the transition zone(s) have a width in r-squared space where the transition zone width in r-squared space may be less than 15% of the width in r-squared space of the echelette. The width in r-squared space of the transition zone may be greater than 5% of the width in r-squared space of the echelette.

The transition zone may define a step height of the at least one echelette. In addition, the optical zone may have a parabolic shape. Further, the optical zone and the transition zone may both have a linear shape in r-squared space.

The diffractive profile includes a plurality of echelettes, each echelette having an optical zone and a transition zone, the transition zone having a parabolic shape. The transition zones of the diffractive profile may join each of the plurality of echelettes together. The diffractive profile may include at least three of the plurality of echelettes. It is further envisioned that each optical zone may have a parabolic shape.

Embodiments herein described include a method comprising fabricating an optic for an ophthalmic lens, the optic including a diffractive profile including at least one echelette having an optical zone and a transition zone, the transition zone having a parabolic shape. The method may further include receiving an ophthalmic lens prescription, and fabricating the optic based on the ophthalmic lens prescription. The method may further include determining one or more of the diffractive profile or a refractive profile of the optic based on the ophthalmic lens prescription. The fabricated optic may have an optical zone that is parabolic in shape. The echelette(s) may have a width in r-squared space and the transition zone a width in r-squared space that is less than 15% of the width in r-squared space of the echelette. Further, the width in r-squared space of the transition zone may be greater than 5% of the width in r-squared space of the echelette.

Embodiments herein described include a system for fabricating an ophthalmic lens. The system may include a processor configured to determine a diffractive profile of an optic, the diffractive profile including at least one echelette having an optical zone and a transition zone, the transition zone having a parabolic shape. The system may include a manufacturing assembly that fabricates the optic based on the diffractive profile.

The system may further include an input for receiving an ophthalmic lens prescription, and the processor may be configured to determine one or more of the diffractive profile or a refractive profile of the optic based on the ophthalmic lens prescription. The optic may have an optical zone that is parabolic in shape. The echelette(s) may have a width in r-squared space and the transition zone a width in r-squared space that is less than 15% of the width in r-squared space of the echelette. Further, the width in r-squared space of the transition zone may be greater than 5% of the width in r-squared space of the echelette.

### BRIEF DESCRIPTION OF THE DRAWINGS

FIG. 1A illustrates a cross-sectional view of an eye with an implanted multifocal refractive intraocular lens.

FIG. 1B illustrates a cross-sectional view of an eye having an implanted multifocal diffractive intraocular lens.

FIG. 2A illustrates a front view of a diffractive multifocal intraocular lens.

FIG. 2B illustrates a cross-sectional view of a diffractive multifocal intraocular lens.

FIGS. 3A-3B are graphical representations of a portion of the diffractive profile of a conventional diffractive multifocal lens.

FIG. 4 illustrates a graph of halo intensity for a parabolic optic having a circular transition compared to a cosine optic having a sine transition.

FIG. 5 illustrates a graph of modulation transfer function (MTF) for a parabolic optic having a circular transition compared to a cosine optic having a sine transition.

FIG. 6 illustrates a graph of halo intensity for a parabolic optic having a sine transition compared to a cosine optic having a sine transition.

FIG. 7 illustrates a graph of modulation transfer function (MTF) for a parabolic optic having a sine transition compared to a cosine optic having a sine transition.

FIG. 8A illustrates a graphical representation of a portion of a diffractive profile according to an embodiment of the present disclosure.

FIG. 8B illustrates a graphical representation of the portion of the diffractive profile shown in FIG. 8A in r-squared space.

FIG. 8C illustrates a graphical representation of a portion of a diffractive profile.

FIG. 9 illustrates a graph of halo intensity for a parabolic optic having a parabolic transition compared to a parabolic optic having a sine transition.

FIG. 10 illustrates a graph of modulation transfer function (MTF) for a parabolic optic having a parabolic transition compared to a parabolic optic having a sine transition.

FIG. 11 illustrates an embodiment of a system.

### DETAILED DESCRIPTION

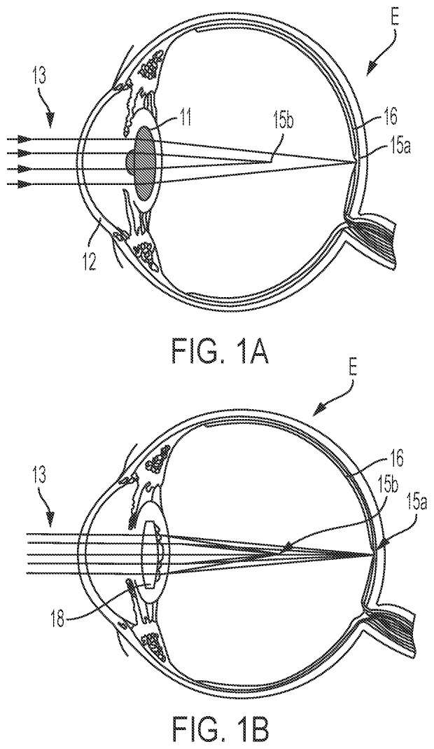

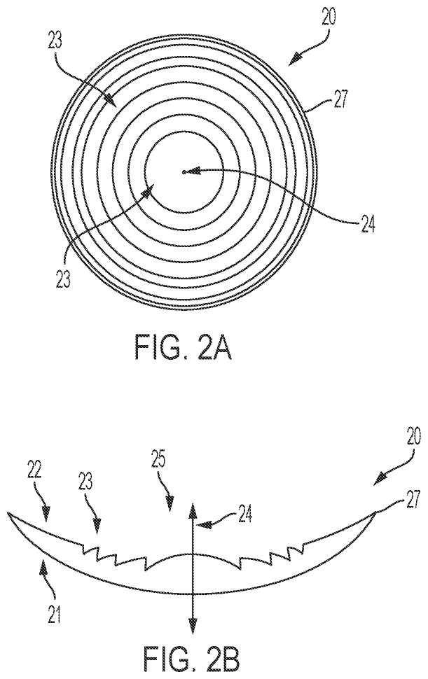

FIGS. 1A, 1B, 2A, 2B, 3A and 3B illustrate multifocal IOL lens geometries, aspects of which are described in U.S. Patent Publication No. 2011-0149236 A1, which is hereby incorporated by reference in its entirety.

FIG. 1A is a cross-sectional view of an eye E fit with a multifocal IOL 11. As shown, multifocal IOL 11 may, for example, comprise a bifocal IOL. Multifocal IOL 11 receives light from at least a portion of cornea 12 at the front of eye E and is generally centered about the optical axis of eye E. For ease of reference and clarity, FIGS. 1A and 1B do not disclose the refractive properties of other parts of the eye, such as the corneal surfaces. Only the refractive and/or diffractive properties of the multifocal IOL 11 are illustrated.

Each major face of lens 11, including the anterior (front) surface and posterior (back) surface, generally has a refractive profile, e.g. biconvex, plano-convex, plano-concave, meniscus, etc. The two surfaces together, in relation to the properties of the surrounding aqueous humor, cornea, and other optical components of the overall optical system, define the effects of the lens 11 on the imaging performance by eye E. Conventional, monofocal IOLs have a refractive power based on the refractive index of the material from which the lens is made, and also on the curvature or shape of the front and rear surfaces or faces of the lens. One or more support elements may be configured to secure the lens 11 to a patient's eye.

Multifocal lenses may optionally also make special use of the refractive properties of the lens. Such lenses generally include different powers in different regions of the lens so as to mitigate the effects of presbyopia. For example, as shown in FIG. 1A, a perimeter region of refractive multifocal lens 11 may have a power which is suitable for viewing at far viewing distances. The same refractive multifocal lens 11 may also include an inner region having a higher surface curvature and a generally higher overall power (sometimes referred to as a positive add power) suitable for viewing at near distances.

Rather than relying entirely on the refractive properties of the lens, multifocal diffractive IOLs or contact lenses can also have a diffractive power, as illustrated by the IOL 18 shown in FIG. 1B. The diffractive power can, for example, comprise positive or negative power, and that diffractive power may be a significant (or even the primary) contributor to the overall optical power of the lens. The diffractive power is conferred by a plurality of concentric diffractive zones which form a diffractive profile. The diffractive profile may either be imposed on the anterior face or posterior face or both.

The diffractive profile of a diffractive multifocal lens directs incoming light into a number of diffraction orders. As light 13 enters from the front of the eye, the multifocal lens 18 directs light 13 to form a far field focus 15a on retina 16 for viewing distant objects and a near field focus 15b for viewing objects close to the eye. Depending on the distance from the source of light 13, the focus on retina 16 may be the near field focus 15b instead. Typically, far field focus 15a is associated with 0^{th} diffractive order and near field focus 15b is associated with the 1^{st} diffractive order, although other orders may be used as well.

Bifocal ophthalmic lens 18 typically distributes the majority of light energy into two viewing orders, often with the goal of splitting imaging light energy about evenly (50%:50%), one viewing order corresponding to far vision and one viewing order corresponding to near vision, although typically, some fraction goes to non-viewing orders.

Corrective optics may be provided by phakic IOLs, which can be used to treat patients while leaving the natural lens in place. Phakic IOLs may be angle supported, iris supported, or sulcus supported. The phakic IOL can be placed over the natural crystalline lens or piggy-backed over another IOL. It is also envisioned that the present disclosure may be applied to inlays, onlays, accommodating IOLs, pseudophakic IOLs, other forms of intraocular implants, spectacles, and even laser vision correction.

FIGS. 2A and 2B show aspects of a conventional diffractive multifocal lens 20. Multifocal lens 20 may have certain optical properties that are generally similar to those of multifocal IOLs 11, 18 described above. Multifocal lens 20 has an anterior lens face 21 and a posterior lens face 22 disposed about an optical axis 24. The faces 21, 22, or optical surfaces, extend radially outward from the optical axis 24 to an outer periphery 27 of the optic. The optical axis 24 may pass through a central zone 25 of the optic. The faces 21, 22, or optical surfaces, face opposite each other.

When fitted onto the eye of a subject or patient, the optical axis of lens 20 is generally aligned with the optical axis of eye E. The curvature of lens 20 gives lens 20 an anterior refractive profile and a posterior refractive profile. Although a diffractive profile may also be imposed on either anterior face 21 and posterior face 22 or both, FIG. 2B shows posterior face 22 with a diffractive profile. The diffractive profile is characterized by a plurality of annular diffractive zones or echelettes 23 spaced about optical axis 24. While analytical optics theory generally assumes an infinite number of echelettes, a standard multifocal diffractive IOL typically has at least 9 echelettes, and may have over 30 echelettes. For the sake of clarity, FIG. 2B shows only 4 echelettes. Typically, an IOL is biconvex, or possibly plano-convex, or convex-concave, although an IOL could be plano-plano, or other refractive surface combinations.

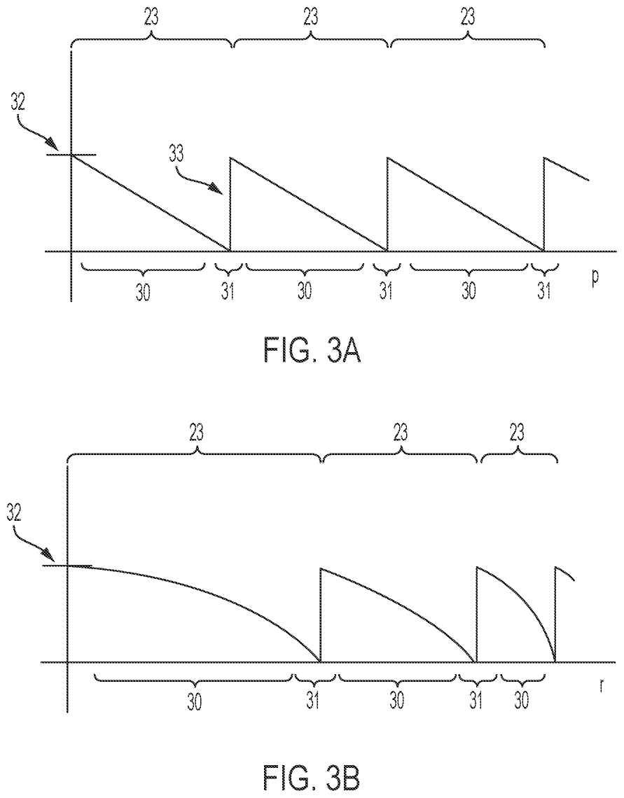

FIGS. 3A and 3B are graphical representations of a portion of a typical diffractive profile of a multifocal lens. While the graph shows only 3 echelettes, typical diffractive lenses extend to at least 9 echelettes to over 32 echelettes. In FIG. 3A, the height 32 of the surface relief profile (from a plane perpendicular to the light rays) of each point on the echelette surface is plotted against the square of the radial distance (r² or p) from the optical axis of the lens (referred to as r-squared space). In multifocal lenses, each echelette 23 may have a diameter or distance from the optical axis which is often proportional to Ain, n being the number of the echelette 23 as counted from optical axis 24. Each echelette has a characteristic optical zone 30 and transition zone 31. Optical zone 30 typically has a shape or downward slope that is parabolic as shown in FIG. 3B. The slope of each echelette in r-squared space (shown in FIG. 3A), however, is the same. As for the typical diffractive multifocal lens, as shown here, all echelettes have the same surface area. The area of echelettes 23 determines the diffractive power of lens 20, and, as area and radii are correlated, the diffractive power is also related to the radii of the echelettes. The step height, or zone height, is the physical vertical offset of the leading edge to the trailing edge of each optical zone. An exemplary step height between adjacent echelettes is marked as reference number 33 in FIG. 3A. The step heights remain the same in r-squared space (FIG. 3A) and in linear space (FIG. 3B). The offset, or step offset, is the height offset of the leading edge from the underlying base curve. The transition zones may join the echelettes together.

A factor contributing to visual symptoms in diffractive lenses are the transition zones between the echelettes. The width of the transition zone may not occur sharply as a single step but may have a gradual transition. For example, the width may be caused by the radius of the manufacturing tool utilized to create the profile. A larger width of a transition zone may result in greater adverse visual symptoms, including halo, glare, and scatter.

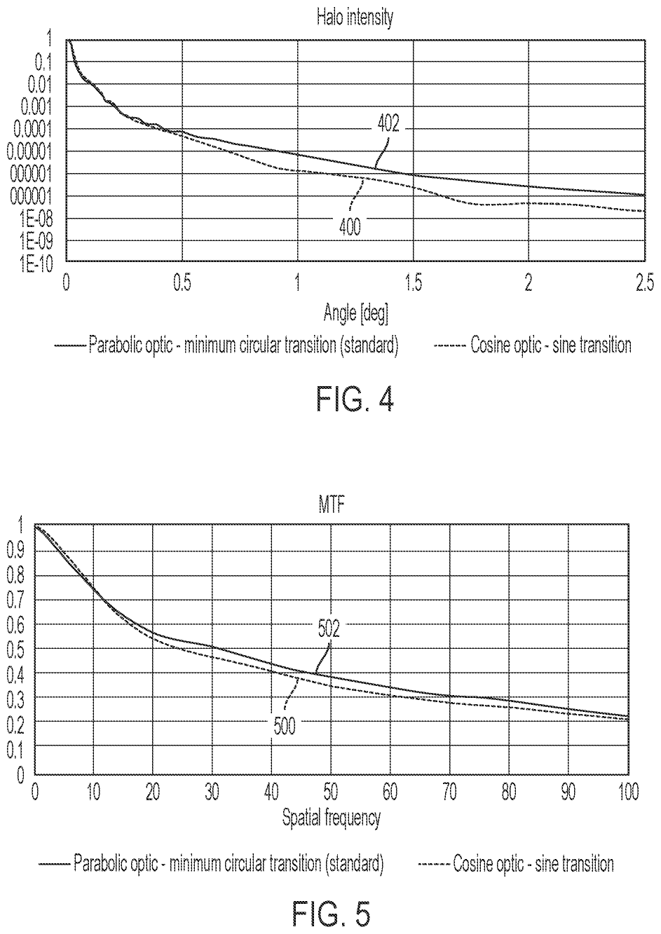

Transition zones may be provided that may reduce the possibility of visual symptoms resulting from the transition zones. Transition zones may be provided, for example, having a sine shape, which may reduce the possibility of visual symptoms. FIG. 4, for example, illustrates a graph of halo intensity in which a diffractive pattern including echelettes that have a cosine optical zone and a sine shaped transition zone, is compared to halo intensity for a diffractive pattern including echelettes having a parabolic optical zone and a transition zone that is circular. The vertical axis shows irradiance (W/mm²) normalized to the peak irradiance. The circular transition zone may be provided by the shape of the tool (e.g., a lathe) utilized to form the transition zone.

Line 400, for example, corresponds to the diffractive pattern including echelettes having a cosine optical zone and a sine shaped transition zone. Line 402 corresponds to the diffractive pattern including echelettes having a parabolic optical zone and a transition zone that is circular. As shown, the halo intensity is reduced for the diffractive pattern including echelettes having a cosine optical zone and a sine shaped transition zone.

The modulation transfer function (MTF) of the diffractive pattern including echelettes having a cosine optical zone and a sine shaped transition zone, however, may be lower than the MTF for the diffractive pattern including echelettes having a parabolic optical zone and a circular transition zone. FIG. 5, for example, illustrates a graph of MTF comparing the line 500 for the diffractive pattern including echelettes having a cosine optical zone and a sine shaped transition zone, with line 502 for the diffractive pattern including echelettes having a parabolic optical zone and a transition zone that is circular.

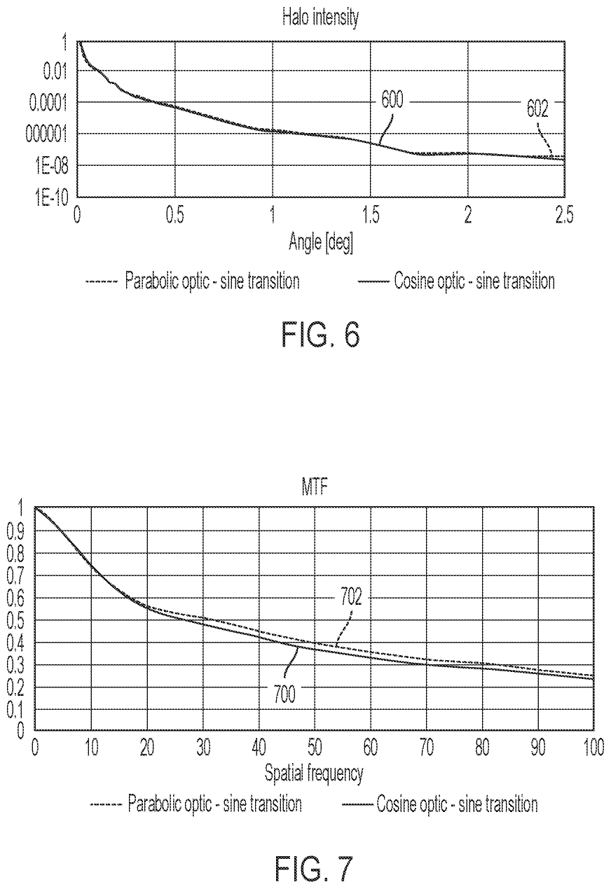

Other shapes of optical zones may improve the MTF for a sine transition. For example, a parabolic optical zone may be provided. FIG. 6 illustrates a graph of halo intensity comparing a line 600 corresponding to a diffractive pattern including echelettes having a cosine optical zone and a sine shaped transition zone, with a line 602 corresponding to a diffractive pattern including echelettes having a parabolic optical zone and a sine shaped transition zone. The halo intensity is shown to be nearly identical, however, the MTF is shown to be improved for the diffractive pattern including echelettes having a parabolic optical zone and a sine shaped transition zone (as reflected in line 702 in FIG. 7) compared to the diffractive pattern including echelettes having a cosine optical zone and a sine shaped transition zone (as reflected in line 700).

However, a transition zone may be provided according to embodiments herein that may provide improvements in reduction of halo intensity, and may otherwise reduce adverse optical effects such as glare and scatter. In embodiments herein, a transition zone having a parabolic shape may be utilized.

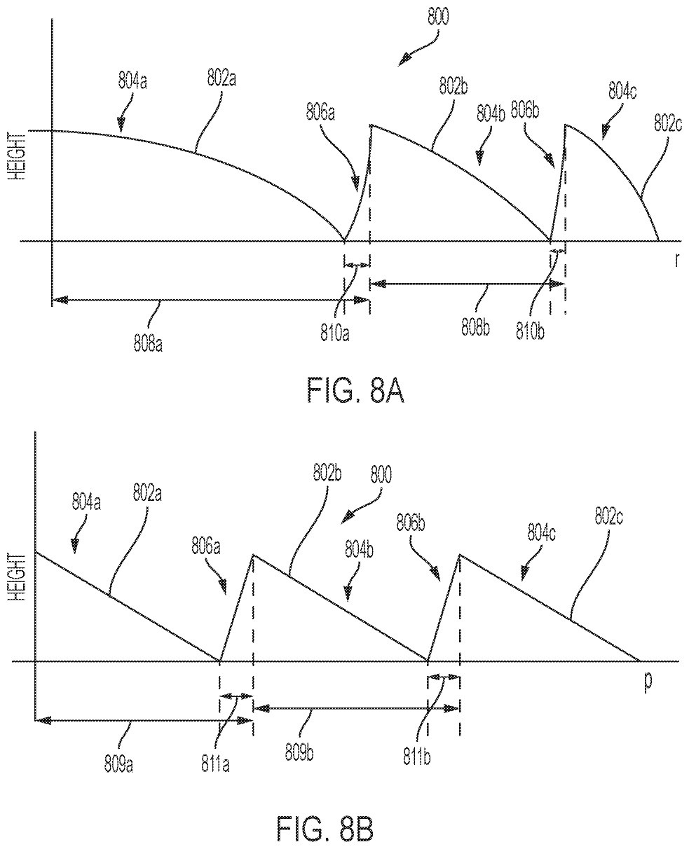

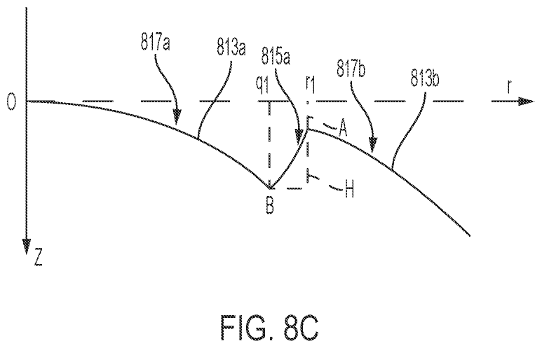

FIG. 8A, for example, illustrates a diffractive profile 800 including a plurality of echelettes 802a-c. As disclosed herein, the number of echelettes utilized may be greater or lesser than three, or may comprise three as shown in FIG. 8A. For example, at least one, at least two, or at least three echelettes, or a greater or lesser number may be utilized as desired. Each echelette 802a-c may include a respective optical zone 804a-c, and may include a respective transition zone (zones 806a and 806b are marked).

Each echelette 802a-c may have a width (respective widths 808a and 808b of echelettes 802a, b are marked). The transition zones may have a width 810a, 810b that may comprise a proportion of the width of the respective echelette (echelette 802a may include transition zone 806a and echelette 802b may include transition zone 806b).

FIG. 8B illustrates the diffractive profile 800 shown in FIG. 8A in r-squared space. The width 808a of the echelette 802a may correspond to the width 809a in r-squared space, and the width 810a of the transition zone 806a may correspond to the width 811a in r-squared space. The width 808b of the echelette 802b may correspond to the width 809b in r-squared space, and the width 810b of the transition zone 806b may correspond to the width 811b in r-squared space. According to embodiments herein, the width 811a, b of the transition zones 806a, b in r-squared space may be less than 15% of the width of the respective echelette 802a, b in r-squared space, and in embodiments may be between 5% and 15% of the width of the respective echelette in r-squared space (i.e., greater than 5% of the width in r-squared space). For example, if the width 809a of the echelette 802a in r-squared space is 1, then the width 811a in r-squared space of the respective transition zone 806a may be less than 0.15. With reference to FIG. 8C, if the width r₁² of the echelette 813a is 1, then r₁²−q₁² would be less than 0.15. In embodiments, other widths may be utilized as desired.

The transition zone 806a, b may define the step height of the respective echelette.

Each transition zone 806a, 806b may have a parabolic shape, and may have a relatively low slope that transitions between the echelettes. For example, referring to FIG. 8C, a representation of two echelettes 813a, b is shown, including a transition zone 815a of the echelette 813a. Respective optical zones 817a, b of the echelettes 813a, b are marked. The horizontal axis is in radius (in linear space). The radius r₁ represents the width of the first echelette 813a (l=1) and the radius q₁ represents the width of the optical zone 817a, or the width of the echelette 813a prior to the transition zone 815a. A height (H) of the transition zone 815a may comprise the height from the lowest height (B) of the transition zone 815a to the greatest height (A) of the transition zone 815a (i.e., H=A−B). The Sag of any transition zone (with the number of the transition zone from the optical axis indicated by l) may be provided by the following equation, in which A and B can be positive or negative and q₁≤r≤r₁.

$$Sag\_transition ( r ) = H \frac{( {r}^{2} - {q}_{l}^{2} )}{( {r}_{l}^{2} - {q}_{l}^{2} )} + B \qquad ( 1 )$$

A parabolic shape for each transition zone may result, which may comprise a linear shape in r-squared space (as shown in FIG. 8B for example). At least one transition zone of the diffractive profile may include a parabolic shape, or in embodiments, a plurality, or all of the transition zones may include a parabolic shape.

The parabolic shape may be configured such that in r-squared space (as shown in FIG. 8B), the transition zones 806a, 806b may return the diffractive profile to the nominal height of the curvature of the optic.

Each optical zone 804a-c may have a parabolic shape. The parabolic shape may be configured such that in r-squared space (as shown in FIG. 8B), each optical zone 804a-c may decrease linearly.

Other configurations of echelettes may be utilized in embodiments as desired. For example, in embodiments, a cosine shape for any or all of the transition zones of the echelettes may be utilized. The echelettes may be utilized in any form of diffractive optic, including an achromatic optic.

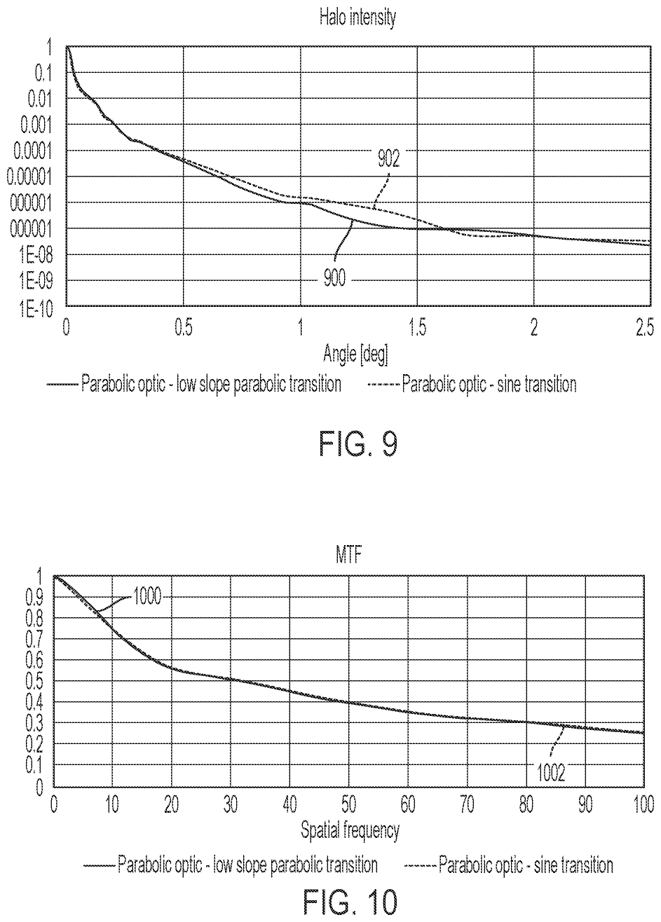

A diffractive profile including echelettes having transition zones with a parabolic shape may provide improved visual results, and decrease adverse optical effects (such as halos, glare, and scatter). FIG. 9, for example, illustrates a graph comparing the halo intensity (marked with line 900) of an embodiment as shown in FIG. 8A, having a transition zone with a parabolic shape and an optical zone having a parabolic shape. The halo intensity of such an embodiment is compared with the halo intensity (marked with line 902) of a diffractive profile including a parabolic optical zone and a sine shaped transition zone. The halo intensity is shown to be reduced for the diffractive profile including a transition zone with a parabolic shape and the optical zone with a parabolic shape.

Further, FIG. 10 illustrates the modulation transfer function (MTF) of the embodiment as shown in FIG. 8A, having a transition zone with a parabolic shape and an optical zone having a parabolic shape (marked with line 1000) as compared with the MTF of a diffractive profile including a parabolic optical zone and a sine shaped transition zone (marked with line 1002). The MTF is shown to be improved for the diffractive pattern having a transition zone with a parabolic shape and an optical zone having a parabolic shape.

An optic for an ophthalmic lens that includes a diffractive profile or refractive profile disclosed herein may be fabricated utilizing a variety of methods. A method may include determining optical aberrations of a patient's eye. Measurements of a patient's eye may be made in a clinical setting, such as by an optometrist, ophthalmologist, or other medical or optical professional. The measurements may be made via manifest refraction, autorefraction, tomography, or a combination of these methods or other measurement methods. The optical aberrations of the patient's eye may be determined.

A determination of the visual range of the patient may also be determined. For example, the ability of the patient to focus on near objects (presbyopia) may be measured and determined. A range of add power for the ophthalmic lens may be determined.

The measurements of the patient's eye may be placed in an ophthalmic lens prescription, which includes features of an optic that are intended to address the optical aberrations of the patient's eye, as well as features that address the visual range for the patient (e.g., an amount of add power and number of focuses to be provided by the optic).

The ophthalmic lens prescription may be utilized to fabricate an optic for the ophthalmic lens. A refractive profile of the optic may be determined based on the ophthalmic lens prescription, to correct for the optical aberrations of the patient's eye. Such a refractive profile may be applied to the optic, whether on a surface including the diffractive profile or on an opposite optical surface. The diffractive profile may also be determined to provide for the desired distribution of add power for the optic.

The determination of one or more of a refractive or diffractive profile and the fabrication of the optic may be performed remotely from the optometrist, ophthalmologist, or other medical or optical professional that performed the measurements of a patient's eye, or may be performed in the same clinical facility of such an individual. If performed remotely, the fabricated optic may be delivered to an optometrist, ophthalmologist, or other medical or optical professional, for being provided to a patient. For an intraocular lens, the fabricated optic may be provided for implant into a patient's eye.

The fabricated optic may be a custom optic fabricated specifically for the patient's eye, or may be fabricated in a manufacturing assembly and then selected by an optometrist, ophthalmologist, or other medical or optical professional for supply to a patient, which may include implantation in the patient's eye.

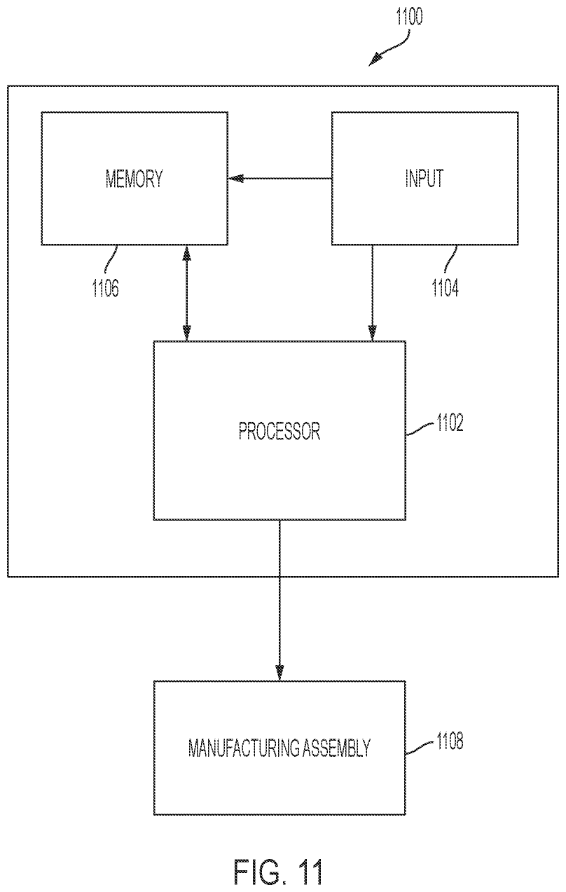

FIG. 11 illustrates an embodiment of a system 1100 that may be utilized to perform all or a portion of the methods disclosed herein. The system 1100 may include a processor 1102, an input 1104, and a memory 1106. In certain embodiments the system 1100 may include a manufacturing assembly 1108.

The processor 1102 may comprise a central processing unit (CPU) or other form of processor. In certain embodiments the processor 1102 may comprise one or more processors. The processor 1102 may include one or more processors that are distributed in certain embodiments, for example, the processor 1102 may be positioned remote from other components of the system 1100 or may be utilized in a cloud computing environment. The memory 1106 may comprise a memory that is readable by the processor 1102. The memory 1106 may store instructions, or features of intraocular lenses, or other parameters that may be utilized by the processor 1102 to perform the methods disclosed herein. The memory 1106 may comprise a hard disk, read-only memory (ROM), random access memory (RAM) or other form of non-transient medium for storing data. The input 1104 may comprise a port, terminal, physical input device, or other form of input. The port or terminal may comprise a physical port or terminal or an electronic port or terminal. The port may comprise a wired or wireless communication device in certain embodiments. The physical input device may comprise a keyboard, touchscreen, keypad, pointer device, or other form of physical input device. The input 1104 may be configured to provide an input to the processor 1102.

The system 1100 may be utilized to perform the methods disclosed herein, such as the processes of determining a diffractive profile of the optic, as well as a refractive profile of the optic. The processor 1102 may be configured to determine the diffractive profile to provide for various add powers for the optic, as well as determining a refractive profile to correct for ocular aberrations of the patient.

The processor 1102 may provide the refractive profile and/or diffractive profile to the manufacturing assembly 1108, which may be configured to fabricate the optic for the ophthalmic lens based on the refractive profile and/or diffractive profile. The manufacturing assembly 1108 may comprise one or more apparatuses for forming the optic, and may comprise a high volume manufacturing assembly or a low volume manufacturing assembly. The manufacturing assembly 1108 may be used for manufacture remote to a clinic in which measurements of the individual's eye or made, or local to such a clinic. The manufacturing assembly may include apparatuses such as lathe tools, or other lens formation devices to fabricate the optic. A tool such as a lathe or other manufacturing apparatus may be utilized to form the parabolic shape for the transition zones if desired. A split-tool may be utilized, which may represent only 5% of the echelette width in embodiments.

In one embodiment, the processor 1102 may be provided with an ophthalmic lens prescription for the individual's eye that may be provided as discussed herein. The processor 1102 may receive the ophthalmic lens via the input 1104. The system 1100 may fabricate the optic for the ophthalmic lens based on the prescription. One or more of a diffractive profile or refractive profile of the optic may be determined based on the ophthalmic lens prescription.

The system 1100 may be configured to fabricate any of the embodiments of ophthalmic lenses disclosed herein.

In one embodiment, a diffractive profile as disclosed herein may be positioned on a surface of a lens that is opposite an aspheric surface. The aspheric surface on the opposite side of the lens may be designed to reduce corneal spherical aberration of the patient.

In one embodiment, one or both surfaces of the lens may be aspherical, or include a refractive surface designed to extend the depth of focus, or create multifocality.

In one embodiment, a refractive zone on one or both surfaces of the lens may be utilized that may be the same size or different in size as one of the diffractive zones. The refractive zone includes a refractive surface designed to extend the depth of focus, or create multifocality.

Any of the embodiments of lens profiles discussed herein may be apodized to produce a desired result. The apodization may result in the step heights and step offsets of the echelettes being gradually varied according to the apodization, as to gradually increasing the amount of light in the distance focus as a function of pupil diameter.

The features of the optics disclosed herein may be utilized by themselves, or in combination with refractive profiles of the optics and/or with features providing for correction of chromatic aberrations (e.g., achromats, which may be diffractive).

The ophthalmic lenses disclosed herein in the form of intraocular lenses are not limited to lenses for placement in the individual's capsular bag. For example, the intraocular lenses may comprise those positioned within the anterior chamber of the eye. In certain embodiments the intraocular lenses may comprise “piggy back” lenses or other forms of supplemental intraocular lenses.

Features of embodiments may be modified, substituted, excluded, or combined as desired.

In addition, the methods herein are not limited to the methods specifically described, and may include methods of utilizing the systems and apparatuses disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An ophthalmic lens comprising:
an optic including a diffractive profile including at least one echelette having an optical zone and a transition zone, the transition zone having a parabolic shape.

2. The ophthalmic lens of claim 1, wherein the at least one echelette has a width in r-squared space and the transition zone has a width in r-squared space that is less than 15% of the width in r-squared space of the at least one echelette.

3. The ophthalmic lens of claim 2, wherein the width in r-squared space of the transition zone is greater than 5% of the width in r-squared space of the at least one echelette.

4. The ophthalmic lens of claim 1, wherein the transition zone defines a step height of the at least one echelette.

5. The ophthalmic lens of claim 1, wherein the optical zone has a parabolic shape.

6. The ophthalmic lens of claim 1, wherein the optical zone and the transition zone both have a linear shape in r-squared space.

7. The ophthalmic lens of claim 1, wherein the diffractive profile includes a plurality of echelettes, each echelette having an optical zone and a transition zone, the transition zone having a parabolic shape.

8. The ophthalmic lens of claim 7, wherein the transition zones of the diffractive profile join each of the plurality of echelettes together.

9. The ophthalmic lens of claim 8, wherein the diffractive profile includes at least three of the plurality of echelettes.

10. The ophthalmic lens of claim 7, wherein each optical zone has a parabolic shape.

11-20. (canceled)
